(19) United States
(12) Patent Application Publication
(10) Pub. No.: US 2026/0260528 A1
(43) Pub. Date: Sep. 3, 2026

(54) ACCESS CONTROL METHOD AND ACCESS CONTROL APPARATUS

(71) Applicant: Telefonaktiebolaget LM Ericsson (publ), Stockholm (SE)

(72) Inventor: Yanping DANG, Beijing (CN)

(21) Appl. No.: 18/879,521

(22) PCT Filed: Sep. 30, 2022
(86) PCT No.: PCT/CN2022/123220

§ 371 Date: Dec. 27, 2024

(51) Int. Cl. G07C 9/22; G07C 9/27

(52) U.S. Cl. CPC G07C 9/22; G07C 9/27

(57) ABSTRACT

Embodiments of the present disclosure provide access control method and access control apparatus. The access control method for controlling access right of a person for entering a place physically may include: obtaining an identity information of the person; retrieving a health related information based on the identity information of the person; comparing the health related information with a preset rule of access right for entering the place physically; and controlling the access right of the person based on the comparison result.

### TECHNICAL FIELD

The non-limiting and exemplary embodiments of the present disclosure generally relate to the technical field of information processing, and specifically to a method and an apparatus for controlling access right of a person for entering a place physically.

### BACKGROUND

This section introduces aspects that may facilitate a better understanding of the disclosure. Accordingly, the statements of this section are to be read in this light and are not to be understood as admissions about what is in the prior art or what is not in the prior art.

Currently, because of COVID-19 epidemic, people need to be checked in regard of his/her health condition when he/she wants to enter a place physically to reduce the possibility of spreading virus. For example, a person needs to have a green (means OK) health code or have a negative nucleic acid test result within 24/48/72 hours to enter into a public place or a place without window, etc. A person who needs to see a doctor with a fever within 7 days is not allowed to enter outpatient building of a hospital, instead, he/she needs to go to fever clinics. The health condition of a person is often checked at the entrance of a building/a room/an area by guards, e.g., security staff or clerks or nurses. They need to stay in close with the visitors and they need to wear masks, or protective suits to protect themselves from virus. This requires man-power and still causes the danger of spreading virus to guards. Secondly, the guards may overlook some visitors when there are too many visitors. Thirdly, the visitors may use false movement, false picture, etc., to fool the guards. Fourthly, the rules for entering a place may change from time to time and guards may make mistakes by using the out-of-date rules. Fifthly, the guards may intentionally pass some visitors whose health condition does not conform to the rules. Sixthly, the speed of checking by human guards are typically slow and it may cause people to gather in a small entrance area which in turn causes virus spreading. Seventhly, some people may not know how to operate a mobile phone for health check and some people may forget to carry a mobile phone, or the mobile phone is out of power.

There is a need to develop a technology of access control that can facilitate the health check at the entrance of a place.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter.

To overcome or mitigate at least one of the above mentioned problems or other problems or provide a useful solution, embodiments of the present disclosure propose a method and an apparatus for controlling access right of a person for entering a place physically.

In a first aspect of the disclosure, there is provided a method for controlling access right of a person for entering a place physically. The method comprises: obtaining an identity information of the person; retrieving a health related information based on the identity information of the person; comparing the health related information with a preset rule of access right for entering the place physically; and controlling the access right of the person based on the comparison result.

In an embodiment, the identity information of the person comprises: a temporal identification or a permanent identification of the person, or a real name associated number.

In an embodiment, the obtaining an identity information of the person comprises: retrieving the identity information of the person through at least one of: biological detection, sweeping a card or chip, scanning a QR code and filling a form; OR receiving the identity information of the person from a device performing the above retrieving step.

In an embodiment, the health related information comprises at least one of: nucleic acid test result, term of validity of nucleic acid test result, health code, travel information, vaccine injection record, whether has a fever in predetermined time period and current temperature.

In an embodiment, wherein the retrieving a health related information based on the identity information of the person comprises: sending a request for the health related information with the identity information of the person to a server; and receiving the health related information from the server.

In an embodiment, the retrieving a health related information based on the identity information of the person comprises: searching for the health related information based on the identity information of the person in a database.

In an embodiment, the preset rule of access right for entering the place physically is stored locally or is received from a server.

In an embodiment, the comparing the health related information with a preset rule of access right for entering the place physically comprises: checking whether the health related information conforms to the preset rule; and determining the access right to be approved if the checked result is positive and determining the access right to be denied if the checked result is negative.

In an embodiment, the controlling the access right of the person based on the comparison result comprises at least one of: displaying the access right through color, light, sound, alarm, voice announcement, text, symbol and/or video; opening or closing the security door for entering the place according to the access right; and sending the access right of the person to a device performing the above displaying or opening/closing step.

In an embodiment, the method further comprises: logging an access attempt of the person to the place, the access right and time.

In a second aspect of the disclosure, there is provided an apparatus for controlling access right of a person for entering a place physically. The apparatus includes: a communication interface; a processor; and a memory coupled to the processor, said memory containing instructions executable by said processor, whereby the apparatus is operative to perform a method according to the first aspect.

In a third aspect of the disclosure, there is provided a computer-readable storage medium storing instructions which when executed by at least one processor, cause the at least one processor to perform the method according to the first aspect.

With the present invention, the health condition of a visitor can be checked accurately, automatically, fast with the updated rules of access control, without manpower usage and the possibility of virus spreading between visitors and guards or among visitors.

### BRIEF DESCRIPTION OF THE DRAWINGS

The above and other aspects, features, and benefits of various embodiments of the present disclosure will become more fully apparent, by way of example, from the following detailed description with reference to the accompanying drawings, in which like reference numerals or letters are used to designate like or equivalent elements. The drawings are illustrated for facilitating better understanding of the embodiments of the disclosure and not necessarily drawn to scale, in which:

FIG. 1 shows a flowchart of a method 100 for controlling access right of a person for entering a place physically according to an embodiment of the present disclosure; and

FIG. 2 is a block diagram of an apparatus 200 for controlling access right of a person for entering a place physically according to embodiments of the present disclosure.

### DETAILED DESCRIPTION

The embodiments of the present disclosure are described in detail with reference to the accompanying drawings. It should be understood that these embodiments are discussed only for the purpose of enabling those skilled persons in the art to better understand and thus implement the present disclosure, rather than suggesting any limitations on the scope of the present disclosure. Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Furthermore, the described features, advantages, and characteristics of the disclosure may be combined in any suitable manner in one or more embodiments. One skilled in the relevant art will recognize that the disclosure may be practiced without one or more of the specific features or advantages of a particular embodiment. In other instances, additional features and advantages may be recognized in certain embodiments that may not be present in all embodiments of the disclosure.

References in the specification to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the associated listed terms.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising”, “has”, “having”, “includes” and/or “including”, when used herein, specify the presence of stated features, elements, and/or components etc., but do not preclude the presence or addition of one or more other features, elements, components and/or combinations thereof.

It is noted that the terms as used in this document are used only for ease of description and differentiation among nodes, devices or networks etc. With the development of the technology, other terms with the similar/same meanings may also be used.

In the following description and claims, unless defined otherwise, all technical and scientific terms used herein have the same meaning as commonly understood by one of ordinary skills in the art to which this disclosure belongs.

An illustrative scenario that embodiments of the present invention can be applied to is a number of people is visiting a place and is being checked for health condition at the entrance of the place. The place may be a building, a floor of the building, an area of the building, a room of the building, etc. For example, the place may be a fever clinic area of a hospital or a non-fever outpatient clinic area of the hospital or an inpatient department of the hospital, or contaminated area or non-contaminated area, etc.

The people may wear a wearable device such as a smart watch, or hold a smart phone, etc. Alternatively, the people may have a ID card, a social security card, a public transportation card, or other cards that is associated with the identity of people. Or, the people can have nothing mentioned above, according to the present invention, they can use their biological characteristics for identification of their identity. Hereinafter, the device that visiting people carries is referred to as “visiting device”. Examples of the visiting device may include at least one of: a wearable device such as a smart watch, smart glasses, a smart health monitoring device, a smart bracelet, a smart wristband; a smart phone, a pad; a dedicated device for visitor health check; a subcutaneous chip; an ID card, a social security card, a driving license card, a public transportation card, etc. At the entrance of the place that people are trying to visit, there is a device referred to as “checking device”. Examples of the checking device may include at least one of: a security door, a door, an attendance machine, a camera, a temperature measurement device, a dedicated device for health check, etc. There is one or more server located anywhere that is connected to the checking device.

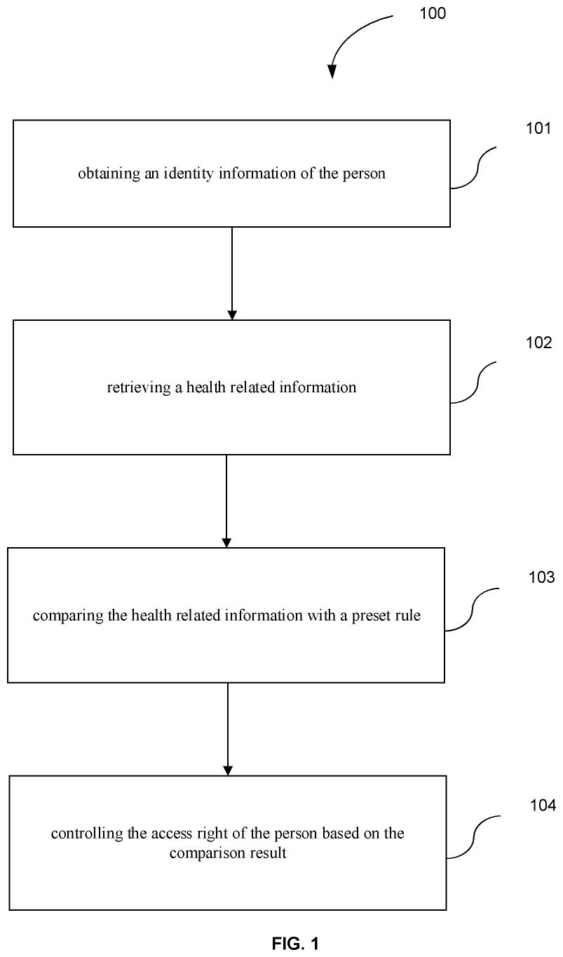

FIG. 1 shows a flowchart of a method 100 for controlling access right of a person for entering a place physically according to an embodiment of the present disclosure.

The method 100 may be implemented in the server, or in the checking device. Alternatively, the method 100 may be partially implemented in the visiting device, partially implemented in the checking device and/or partially implemented in the server.

At block 101, an identity information of the person is obtained.

In an embodiment, the identity information of the person comprises: a temporal identification or a permanent identification of the person, or a real name associated number. The permanent identification of the person may include at least one of: an ID number of the person, a social security number of the person, a driving license number of the person, etc. The temporal identification of the person may include a one-time identity number of the person, a temporal ID number of the person, or any other temporal number that is associated with the person's identity. The real name associated number may include at least one of: a real name mobile phone number, an Integrate circuit card identity (ICCID) number of the real name mobile phone number, a real name Internet of Things (IoT) card number, a real name public transportation card number, a real name device (e.g., smart watch or bracelet, or a dedicated device for visitor health check) number, etc.

This step may be implemented in the visiting device, the checking device and/or the server.

In an embodiment, obtaining an identity information of the person may be done by one of the visiting device, the checking device and/or the server directly. For example, the identity information of the person may be retrieved through biological detection such as face recognition, voice print recognition, fingerprint recognition or iris recognition, sweeping a card such as an ID card, a social security card, a driving license card, a public transportation card, sweeping a chip such as subcutaneous chip, RFID chip or sweeping a virtual card (real name) via NFC/bluetooth, scanning a QR code and filling a form, etc.

In an embodiment, obtaining an identity information of the person may be done by receiving the identity information of the person from a device performing the above retrieving step. For example, the visiting device performs the above retrieving step and the checking device performs the obtaining step by receiving the identity information from the visiting device.

At block 102, a health related information is retrieved based on the identity information of the person.

The health related information may include at least one of: nucleic acid test result, term of validity of nucleic acid test result, health code, travel information, vaccine injection record, whether has a fever in predetermined time period and current temperature.

This step may be implemented in the visiting device, the checking device and/or the server.

In an embodiment, the device performing this step may send a request for the health related information with the identity information of the person to a server or a device which has the health related information. The server or the device has a database that stores the health related information in association with the identity information. After searching its database with the identity information, the server or the device sends back the health related information to the device performing this retrieving step.

In an embodiment, the device performing this step may store the health related information in association with the identity information. It searches for the health related information based on the identity information of the person in a database.

At block 103, the health related information is compared with a preset rule of access right for entering the place physically.

The preset rule of access right for entering the place physically may be updated periodically or on basis of need to the latest rule issued by the government or public health department or other administration office. Different places may have different preset rules at different time. The preset rule of access right for entering the place physically is stored locally or is received from a server.

This step may be implemented in the visiting device, the checking device and/or the server.

The health related information is compared with a preset rule of access right for entering the place physically by: checking whether the health related information conforms to the preset rule; and determining the access right to be approved if the checked result is positive and determining the access right to be denied if the checked result is negative.

At block 104, the access right of the person is controlled based on the comparison result.

This step may be implemented in the visiting device, the checking device and/or the server.

In case the checking device performs this step, the checking device may display the access right through color, light, sound, alarm, voice announcement, text, symbol and/or video; or the checking device may open or close the security door for entering the place according to the access right.

In case the visiting device and/or the server performs this step, it may send the access right of the person to a device performing the above displaying or opening/closing step.

In another embodiment, the method 100 further includes logging an access attempt of the person to the place, the access right and time, e.g., in a database.

Because the method can be performed by machine automatically, it does not require any man-power like guards and guards are not subjected to the danger of being infected. Because the method is performed by machine with the latest rules strictly, overlook or being fooled, or mistake can be avoided. Because the method can be performed by machine, speed of check is quicker than check by man and gathering of visitors can be avoided. User experience can be improved.

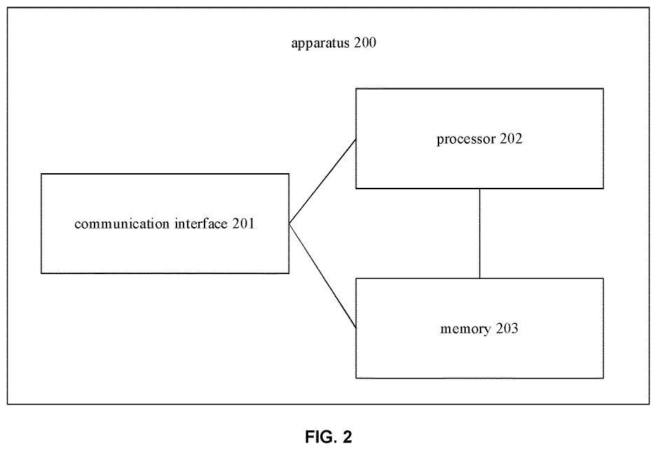

FIG. 2 is a block diagram of an apparatus for controlling access right of a person for entering a place physically according to embodiments of the present disclosure.

The apparatus 200 includes a communication interface 201, a processor 202 and a memory 203. The memory 203 contains instructions executable by the processor 202 whereby the apparatus 200 is operative to perform the actions, e.g., of the procedure described earlier in conjunction with FIG. 1.

In some embodiments, the memory 203 may further contain instructions executable by the processor 202 whereby the apparatus 200 is operative to perform any of the aforementioned methods, steps, and processes.

The present disclosure also provides at least one computer program product in the form of a non-volatile or volatile memory, e.g., a non-transitory computer readable storage medium, an Electrically Erasable Programmable Read-Only Memory (EEPROM), a flash memory and a hard drive. The computer program product includes a computer program. The computer program includes: code/computer readable instructions, which when executed by the processor 202 causes the apparatus 200 to perform the actions, e.g., of the procedure described earlier in conjunction with FIG. 1.

The computer program product may be configured as a computer program code structured in computer program modules. The computer program modules could essentially perform the actions of the flow illustrated in FIG. 1.

The processor may be a single CPU (Central processing unit), but could also comprise two or more processing units. For example, the processor may include general purpose microprocessors; instruction set processors and/or related chips sets and/or special purpose microprocessors such as Application Specific Integrated Circuit (ASICs). The processor may also comprise board memory for caching purposes. The computer program may be carried by a computer program product connected to the processor. The computer program product may comprise a non-transitory computer readable storage medium on which the computer program is stored. For example, the computer program product may be a flash memory, a Random-access memory (RAM), a Read-Only Memory (ROM), or an EEPROM, and the computer program modules described above could in alternative embodiments be distributed on different computer program products in the form of memories.

The techniques described herein may be implemented by various means so that an apparatus implementing one or more functions of a corresponding apparatus described with an embodiment comprises not only prior art means, but also means for implementing the one or more functions of the corresponding apparatus described with the embodiment and it may comprise separate means for each separate function or means that may be configured to perform two or more functions. For example, these techniques may be implemented in hardware (one or more apparatuses), firmware (one or more apparatuses), software (one or more modules), or combinations thereof. For a firmware or software, implementation may be made through modules (e.g., procedures, functions, and so on) that perform the functions described herein.

Exemplary embodiments herein have been described above with reference to block diagrams and flowchart illustrations of methods and apparatuses. It will be understood that each block of the block diagrams and flowchart illustrations, and combinations of blocks in the block diagrams and flowchart illustrations, respectively, can be implemented by various means including computer program instructions. These computer program instructions may be loaded onto a general purpose computer, special purpose computer, or other programmable data processing apparatus to produce a machine, such that the instructions which execute on the computer or other programmable data processing apparatus create means for implementing the functions specified in the flowchart block or blocks.

Further, while operations are depicted in a particular order, this should not be understood as requiring that such operations be performed in the particular order shown or in sequential order, or that all illustrated operations be performed, to achieve desirable results. In certain circumstances, multitasking and parallel processing may be advantageous. Likewise, while several specific implementation details are contained in the above discussions, these should not be construed as limitations on the scope of the subject matter described herein, but rather as descriptions of features that may be specific to particular embodiments. Certain features that are described in the context of separate embodiments may also be implemented in combination in a single embodiment. Conversely, various features that are described in the context of a single embodiment may also be implemented in multiple embodiments separately or in any suitable sub-combination.

While this specification contains many specific implementation details, these should not be construed as limitations on the scope of any implementation or of what may be claimed, but rather as descriptions of features that may be specific to particular embodiments of particular implementations. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

It will be obvious to a person skilled in the art that, as the technology advances, the inventive concept can be implemented in various ways. The above described embodiments are given for describing rather than limiting the disclosure, and it is to be understood that modifications and variations may be resorted to without departing from the spirit and scope of the disclosure as those skilled in the art readily understand. Such modifications and variations are considered to be within the scope of the disclosure and the appended claims. The protection scope of the disclosure is defined by the accompanying claims.

1. An access control method for controlling access right of a person for entering a place physically, comprising:
obtaining an identity information of the person;
retrieving a health related information based on the identity information of the person;
comparing the health related information with a preset rule of access right for entering the place physically; and
controlling the access right of the person based on the comparison result.

2. The method of claim 1, wherein the identity information of the person comprises: a temporal identification or a permanent identification of the person, or a real name associated number.

3. The method of claim 1, wherein the obtaining an identity information of the person comprises:
retrieving the identity information of the person through at least one of: biological detection, sweeping a card or chip, scanning a QR code and filling a form; OR
receiving the identity information of the person from a device performing the above retrieving step.

4. The method of claim 1, wherein the health related information comprises at least one of: nucleic acid test result, term of validity of nucleic acid test result, health code, travel information, vaccine injection record, whether has a fever in predetermined time period and current temperature.

5. The method of claim 1, wherein the retrieving a health related information based on the identity information of the person comprises:
sending a request for the health related information with the identity information of the person to a server; and
receiving the health related information from the server.

6. The method of claim 1, wherein the retrieving a health related information based on the identity information of the person comprises:
searching for the health related information based on the identity information of the person in a database.

7. The method of claim 1, wherein the preset rule of access right for entering the place physically is stored locally or is received from a server.

8. The method of claim 1, wherein the comparing the health related information with a preset rule of access right for entering the place physically comprises:
checking whether the health related information conforms to the preset rule; and
determining the access right to be approved if the checked result is positive and determining the access right to be denied if the checked result is negative.

9. The method of claim 1, wherein the controlling the access right of the person based on the comparison result comprises at least one of:
displaying the access right through color, light, sound, alarm, voice announcement, text, symbol and/or video;
opening or closing the security door for entering the place according to the access right; and
sending the access right of the person to a device performing the above displaying or opening/closing step.

10. The method of claim 1, further comprising:
logging an access attempt of the person to the place, the access right and time.

11. An access control apparatus for controlling access right of a person for entering a place physically, comprising:
a communication interface;
a processor; and
a memory coupled to the processor, said memory containing instructions executable by said processor to perform operations comprising to:
obtain an identity information of the person;
retrieve a health related information based on the identity information of the person;
compare the health related information with a preset rule of access right for entering the place physically; and
control the access right of the person based on the comparison result.

12. A non-transitory computer-readable storage medium storing instructions which when executed by at least one processor, cause the at least one processor to perform operations comprising to:
obtain an identity information of the person;
retrieve a health related information based on the identity information of the person;
compare the health related information with a preset rule of access right for entering the place physically; and
control the access right of the person based on the comparison result.
